(19) United States
(12) Patent Application Publication
(10) Pub. No.: US 2019/0032099 A1
(43) Pub. Date: Jan. 31, 2019

(54) ENZYME-ASSISTED BIO-BASED FIBER GUM COMPOSITION AND PRODUCTION PROCESS

(71) Applicant: The United States of America, as represented by the Secretary of Agriculture., Washington, DC (US)

(72) Inventor: David Johnston, Wyndmoor, PA (US)

(21) Appl. No.: 15/658,808

(22) Filed: Jul. 25, 2017

(51) Int. Cl. C12P 19/04; C08B 37/00; C12P 19/20; C12P 19/14; A23L 29/20

(52) U.S. Cl. CPC C12P 19/04; C08B 37/0057; C12P 19/20; C12Y 302/01003; C12Y 302/01001; A23V 2002/00; C12Y 302/01004; C12Y 302/01021; C12P 19/14; A23L 29/20; C12Y 302/01008

(57) ABSTRACT

Compositions comprising a bio-based fiber gum product subjected to an enzymatic process and methods for producing bio-based fiber gum compositions from bio-based fiber feedstock are disclosed. The methods include subjecting the bio-based fiber feedstock to an enzymatic processes through a series of pH and temperature adjustments to increase efficiency in the production of bio-based fiber gum from bio-based fiber feedstocks. The enzymatic processes include a starch-degrading enzymatic component and a cell wall enzymatic degrading component.

### FIELD OF THE INVENTION

The disclosed invention relates generally to novel and improved bio-based fiber gum compositions and methods of bio-based fiber gum production. More specifically, the invention relates to the utilization of enzymatic treatments, in combination with pH and temperature modifications, to improve the production of bio-based fiber gum compositions by altering the water binding properties of the insoluble cellulosic material and improving solid liquid separation and yield of bio-based fiber gum.

### BACKGROUND OF THE INVENTION

Bio-based fibers include, for example, fibrous portions of agricultural materials including commodities such as oat, corn, sorghum, and wheat. Such fibrous feedstocks contain arabinoxylans, which are cell wall polysaccharides abundant in plants of the family Poaceae. The structural commonality of this class of polysaccharides is the β-(1,4) linked d-xylopyranose backbone with α-1-arabinofuranose side chains linked to O-2 and/or O-3 positions of the xylose residues. A large degree of structural heterogeneity is imparted by the presence of other sugars, including galactose, glucuronic acid, and xylose in the branches. Other non-carbohydrate compounds, such as proteins, lipids, and phenolic acids are often strongly associated or covalently linked to the polysaccharide molecules (Yadav, M. P., et al., Journal of Agricultural and Food Chemistry, 55(3): 943-947 (2007)). Corn fiber arabinoxylan, also called hemicellulose B, for example, is traditionally isolated from the fibrous portions (e.g., pericarp, tip cap, and endosperm cell wall fractions) of corn kernels by alkaline solution extraction, often in the presence of hydrogen peroxide. This isolated corn fiber arabinoxylan is commonly referred to as “corn fiber gum” or “CFG” (Yadav, M. P., et al., Food Hydrocolloids, 23(6): 1488-1493 (2009)).

Corn fiber is typically a byproduct of wet milling, which is the industrial process that produces starch, sweeteners, fuel grade ethanol, and other products from corn. The complex structure of arabinoxylans varies greatly by source, with rice and sorghum arabinoxylans having simple structures (e.g., widely distributed, single sugar arabinose branches) (Rose, D. J., et al., Journal of Agricultural and Food Chemistry, 58(1): 493-499 (2009); Verbruggen, M. A., et al., Carbohydrate Research, 306(1-2): 275-282 (1998)) and corn bran arabinoxylans typically having highly branched and more complex structures (Huisman, M. M. H., et al., Schols, Carbohydrate Polymers, 43: 269-279 (2000); Rumpagaporn, P., et al., Carbohydrate Polymers, 130: 191-197 (2015); Saulnier, L., et al., Carbohydrate Polymers, 26: 279-287 (1995)). Current industry data suggests that the corn processing industry produces about 4 million tons of corn fiber each year, which is generally sold as corn gluten feed, a low-cost ingredient in cattle rations. Corn fiber gum is a water-soluble polymer with functional properties useful in, for example, foods as an emulsifier, soluble dietary fiber, and industrial applications including adhesives and water-based paint thickeners, among others.

Existing methods for isolation of corn fiber gum require multiple operations and also produce an insoluble cellulosic arabinoxylan (CAX) fraction that is inefficient and costly to handle as well as binds large quantities of water. This wet material typically needs to be washed to prevent loss of the corn fiber gum product and must also be further processed for proper disposal. Such washing and processing adds additional cost to the corn fiber gum production process. The recovery of corn fiber gum becomes more complicated due to the high water binding properties of this insoluble fraction. CAX can hold as much as 15× its weight in water, and sheering processes (e.g., blending, high-speed mixing, pumping through an orifice) are commonly used causing increased water binding. In order to minimize loss of usable CFG, the CAX must be extensively washed, resulting in significant dilution of the CFG extract.

The alkaline extraction of corn fiber for the production of corn fiber gum and the production of functionalized insoluble fiber has been previously reported (see e.g., Doner, L W, et al., Isolation of Hemicellulose from Corn Fiber by Alkaline Hydrogen Peroxide Extraction. Cereal Chem., 1997, 74, 176-181; Inglett, G. E., Development of a Dietary Fiber Gel for Calorie-Reduced Foods, Cereal Food World 1997, 42, 382-385; Inglett, G. E., et al., Cellulosic Fiber Gels Prepared from Cell Walls of Maize Hulls. Cereal Chem 2001, 78, 471-475). An exemplary process utilizes a sequential extraction process that first removes the residual starch using an alpha-amylase and extracts the de-starched fiber using alkali (see e.g., Doner, L. W., et al., An Improved Process for Isolation of Corn Fiber Gum, Cereal Chem 1998, 75, 408-411). The alkali extraction process also isolates an insoluble cellulosic arabinoxylan with yields about 35% of the starting de-starched fiber (see e.g., Doner, L. W., et al., Isolation and Characterization of Cellulose/Arabinoxylan Residual Mixtures from Corn Fiber Gum Processes, Cereal Chem 2001, 78, 200-204). An alkali extraction process was developed and subsequently commercialized, that utilizes the insoluble cellulosic material as a fiber for use in food and industrial products. In this process, the insoluble cellulosic material is isolated and functionalized by incorporating a sheering process. The sheering process resulted in the insoluble cellulosic fiber having significantly improved water-binding properties, which is beneficial when such materials are used as a fiber in food and industrial products.

The conversion of corn fiber into monosaccharides for ethanol production showed that corn fiber was extremely recalcitrant to hydrolysis by enzymes (see e.g., Dien, B. S., et al., Fermentation of “quick fiber” produced from a modified corn-milling process into ethanol and recovery of corn fiber oil. Appl Biochem Biotech 2004, 113-16, 937-949; Dien, B. S., et al., Hydrolysis and fermentation of pericarp and endosperm fibers recovered from enzymatic corn dry-grind process. Cereal Chem 2005, 82, 616-620). Pretreatment processes that significantly help improve the conversion of the fiber were developed using both acidic and basic systems (see e.g., Dien, B. S., et al., Chemical composition and response to dilute-acid pretreatment and enzymatic saccharification of alfalfa, reed canary grass, and switchgrass. Biomass Bioenerg 2006, 30, 880-891; Dien, B. S., et al., Enzyme characterization for hydrolysis of AFEX and liquid hot-water pretreated distillers' grains and their conversion to ethanol. Bioresource Technology 2008, 99, 5216-5225; Gould, J. M.; Freer, S. N., High-Efficiency Ethanol-Production from Lignocellulosic Residues Pretreated with Alkaline H₂O₂, Biotechnol Bioeng, 1984, 26, 628-631). It was also observed that acid treatments reduced the arabinose content of CFG significantly and likely altered its functionality (see e.g., Feher, C., et al., Investigation of selective arabinose release from corn fiber by acid hydrolysis under mild conditions. Journal of Chemical Technology and Biotechnology 2015, 90, 896-906; Nghiem, N. P., et al., Fractionation of corn fiber treated by soaking in Aqueous Ammonia (SAA) for isolation of hemicellulose B and production of C5 sugars by enzyme hydrolysis. Appl Biochem Biotech 2011, 164, 1390-1404). High concentrations of enzymes after ammonia pretreatment (basic), could also be used extract very small amounts of arabinoxylan polymers from the corn fiber.

Prior research with cell wall degrading enzymes (e.g., xylanases, cellulases, hemicellulases, beta-glucanases) demonstrated that certain enzymes or mixtures could be applied to alter the water binding properties of cell wall material. In the corn to ethanol process, for example, it was demonstrated that a small amount of water could be released from the insoluble fiber fraction. It was also shown that treatments of cell wall degrading enzymes during fermentation had a significant impact on the water binding properties of corn fiber in the ethanol process (see e.g., Henriques, A. B., et al., Enhancing water removal from whole stillage by enzyme addition during fermentation. Cereal Chem 2008, 85, 685-688; Henriques, A. B., et al., Reduction in energy usage during dry grind ethanol production by enhanced enzymatic dewatering of whole stillage: Plant trial, process model, and economic analysis. Industrial Biotechnology 2011, 7, 288-297). This work also demonstrated that the separation of the liquid phase could be improved using enzymatic treatment as well as potential energy savings and a reduction in water utilization.

There thus exists an industrial need to develop improved methods of efficiently and economically extracting and purifying bio-based fiber gum for use in applications such as emulsifiers, soluble dietary fiber, films, and industrial applications including adhesives, binders, and water-based paint thickeners, among others.

### SUMMARY OF THE INVENTION

To address these challenging issues in bio-based fiber gum production, the present invention accordingly provides novel bio-based fiber gum compositions and methods of using enzymes to alter the water binding properties of the insoluble cellulosic arabinoxylan fraction of the bio-based fiber gum production processes. Using selected enzymes in the disclosed methods, it was surprisingly discovered that the yield of bio-based fiber gum could be significantly improved over conventional extraction processes. Additionally, it was also surprisingly discovered that a substantial portion of the insoluble cellulosic arabinoxylan fraction could be converted into additional bio-based fiber gum.

In an aspect, the invention provides compositions comprising a bio-based fiber gum product subjected to an enzymatic process to reduce an insoluble fraction by at least about 35% as compared a bio-based fiber gum product not subjected to the enzymatic process. In a further aspect, the invention provides processes for producing a bio-based fiber gum from a bio-based fiber feedstock. The processes include subjecting a fiber to a process to create a slurry and adjusting the pH of the slurry to create a pH-adjusted slurry. A starch-degrading enzymatic component is added to the pH-adjusted slurry to create an enzyme-treated slurry, which is incubated at a temperature and time sufficient to create an enzyme-degraded slurry. The pH of the enzyme-degraded slurry is adjusted to create a pH-adjusted enzyme-degraded slurry, which is then incubated at a temperature and time sufficient to create an intermediate product. The intermediate product is then cooled and pH-adjusted to create a cooled intermediate product and a cell-wall degrading enzymatic preparation is added to create a cooled intermediate CWD product. Glucoamylase is optionally added to the cooled intermediate product or the cooled intermediate CWD product along with the cell-wall degrading enzymatic preparation. A degraded product is formed upon incubating the cooled CWD product at a temperature and time sufficient for the cell-wall degrading enzymatic preparation to at least partially or fully degrade the mixture. The bio-based fiber gum product is then recovered through a recovery process.

In another aspect, the invention provides processes for producing a bio-based fiber gum from a bio-based fiber feedstock. The processes include subjecting a fiber to a process to create a slurry and incubating the slurry to create a pretreated slurry. The pH of the pretreated slurry is adjusted to create a pH-adjusted pretreated slurry. An enzymatic cocktail including at least one amylase, at least one cell wall degrading enzyme, and optionally glucoamylase to the pH-adjusted pretreated slurry to create an enzymatic cocktail-treated slurry which is further incubated to create an intermediate product. The pH of the intermediate product is adjusted to create a degraded product which is then subjected to a recovery process to recover the bio-based fiber gum product.

It is an advantage of the invention to provide methods of producing functional bio-based fiber gum with improved yields over previously known processes.

It is another advantage of the present invention to provide methods of efficiently solubilizing bio-based fiber gum from insoluble cellulosic arabinoxylan to improve recovery of bio-based fiber gum with a concomitant decrease in the production of solid waste.

It is a further advantage of the present invention to provide new methods of producing bio-based fiber gum useful to food and corn processors in the development of economically and commercially viable processes for production of food grade bio-based fiber gum.

It is yet another advantage of the present invention to provide methods of efficiently and cost-effectively producing additional bio-based fiber gum from insoluble cellulosic arabinoxylan waste products of the conventional bio-based fiber gum production process.

It is another advantage of the present invention to provide methods of producing higher concentrations of solubilized bio-based fiber thereby reducing the amount of water used in the production process and concomitantly reducing the amount of water that needs to be removed in order to produce the final product.

This summary is provided to introduce a selection of concepts in a simplified form that are further described below in the detailed description. This summary is not intended to identify all key or essential features of the claimed subject matter, nor is it intended as an aid in determining the scope of the claimed subject matter.

### BRIEF DESCRIPTION OF THE DRAWINGS

FIG. 1 is a flow chart showing an embodiment of the processes used in the methods of the invention incorporating the disclosed enzymatic treatments.

FIG. 2 is a flow chart showing an embodiment of the processes used in the methods of the invention incorporating the disclosed enzymatic treatments.

FIG. 3 shows an embodiment of the CFG recovery process for small or large scale recovery of CFG.

FIG. 4 shows an embodiment of the CFG recovery process for small or large scale recovery of CFG.

FIG. 5 shows an embodiment of the CFG recovery process for small or large scale recovery of CFG.

FIG. 6 shows an example of the reduction in pellet volume of CAX after treatment with different cell-wall degrading enzymes overnight at pH 5.5 and 50° C. Letters represent the particular enzyme used and the number is the dosage in μL: GC 220 (A), Multifect GC (B), Accellerase 1500 (C), GC 440 (D), Accellerase XY (E), Accellerase XC (F), Accellerase BG (G), GC Extra (H), Spezyme CP (I) and Multifect Xylanase (J). Control shows the untreated incubated sample. Photo inset shows centrifuged CAX slurry with and without enzyme treatment.

FIG. 7 shows an example of the reduction in the pellet volume of CAX, after enzymatic treatment using GC 220 for 6 hours at pH 5.5 and 50° C.

FIG. 8 is an exemplary chromatogram of hydrolysates from corn fiber gum (CFG), enzymatic isolated corn fiber gum (E CFG) and enzymatically hydrolyzed cellulosic arabinoxylan (E CAX). The refractive index (RI) detector signal has been offset for clarity and to highlight similarity between profiles.

### DETAILED DESCRIPTION OF THE INVENTION

Unless herein defined otherwise, all technical and scientific terms used herein generally have the same meaning as commonly understood by one of ordinary skill in the art to which this invention belongs. The definitions below may or may not be used in capitalized as well as singular or plural form herein and are intended to be used as a guide for one of ordinary skill in the art to make and use the invention and are not intended to limit the scope of the invention. Mention of trade names or commercial products herein is solely for the purpose of providing specific information or examples and does not imply recommendation or endorsement of such products.

“Alpha Amylase” means a starch-degrading enzyme (e.g., systematic name: 4-alpha-D-glucan glucanohydrolase; EC 3.2.1.1) which catalyzes the degradation of various starches to maltose via hydrolyzing bonds between repeating glucose units.

“Bio-Based Fiber” means fibrous portions of at least one agricultural material, such as, for example, corn kernels, sorghum grains, wheat hulls, the like, and combinations thereof.

“Bio-Based Fiber Gum” means the arabinoxylan polymers found in plant cell walls and isolated from the fibrous portions of plants for use in a variety of industrial applications.

“Corn Fiber” means the fibrous portions of corn kernels including pericarp, tip cap, and endosperm cell wall fractions as an individual component or any combination of the components.

“Corn Fiber Gum” or “CFG” means the arabinoxylan polymers (e.g., a copolymer of arabinose and xylose), also called hemicellulose B, commonly found in plant cell walls and isolated from the fibrous portions (e.g., pericarp, tip cap, and endosperm cell wall fractions) of corn kernels for industrial use in a variety of industrial applications.

“CWD Enzyme” or “Cell Wall Degrading Enzyme” means any enzyme capable of depolymerizing or degrading the components of a plant cell wall (e.g., cellulose, hemicellulose, pectin, and other polysaccharides) such as glucanases, chitinases, xylanases, endocellulases, exocellulases, pectinases, polygalacturonases, the like, and any mixture or combination thereof. Commercially available examples of such enzymes include those available from DuPont Industrial Biosciences (Spezyme CP, GC 220, GC 440, GC 880, Multifect® Xylanase, Multifect® GC, Multifect® GC extra, Accelerase® 1500, Accelerase® XY, Accelerase® XC, Accelerase® BG, Optimash® Barley, and IndiAge® Super L as well as Cellic CTec 2 (available from Novozymes) and Viscozyme (available from MilliporeSigma).

“Insoluble Cellulosic Arabinoxylan” or “CAX” means the fraction or secondary product of corn fiber that is not soluble in water during conventional CFG extraction processes.

“Glucoamylase” means an amylase that cleaves the last alpha-1,4-glycosidic linkages at the non-reducing end of amylase and amylopectin to yield glucose. Also known as amyloglucosidase, it cleaves a free glucose molecule from, for example, starch or maltooligosaccharides. In some instances, this enzyme may be interchangeable or complementary to starch-degrading enzymes as disclosed herein.

“Starch Degrading Enzyme” means an enzyme which catalyzes the degradation of various starches to, for example, maltose via hydrolyzing bonds between repeating sugar (e.g., glucose) units. Commercially available examples include SPEZYME® RSL/Alpha/CL (alpha-amylases available from DuPont Industrial Biosciences), OPTIDEX™ L-400 (a glucoamylase available from DuPont Industrial Biosciences), and Liquozyme® SC (an alpha-amylase available from Novozymes).

The present invention demonstrates that a highly soluble, functional bio-based fiber gum may be produced from bio-based fiber with improved yields over previously known processes. More particularly, this invention relates to processes utilizing enzymatic treatments, in combination with pH and temperature modifications, to improve the producing and/or recovery of bio-based fiber gum (e.g., corn fiber gum, sorghum fiber gum, wheat fiber gum, the like, and combinations thereof) by altering the water binding properties of the insoluble cellulosic material fraction and improving solid liquid separation and yield. Though corn feedstock is preferred for the method of the invention, the disclosed methods are applicable to other fibrous feedstocks and agricultural materials. Examples of other fibrous feedstocks that may be used are oat, sorghum, wheat hulls, wheat fiber, the like, and combinations thereof. It has been surprisingly and unexpectedly demonstrated that the secondary CAX product of the CFG extraction process may be efficiently solubilized to improve recovery of CFG and thereby decrease production of solid CAX waste product. The expected result would have been to observe little or no increase in CFG yield. It would also have been anticipated that the enzyme treatment may have damaged the CFG thereby decreasing yields, which was not observed.

A new method for isolation of corn fiber gum that incorporates cell wall hydrolyzing enzymes to remove the insoluble cellulosic material was developed. Multiple enzyme preparations were evaluated for improved yields of corn fiber gum. HPLC analysis of the released sugars from the insoluble cellulosic material was used for enzyme screening and selection (see examples below). Incorporating the enzyme treatment, corn fiber gum yields were substantially and surprisingly improved relative to the conventional non-enzymatic process. Sugar profiles were compared for the different conventional and enzymatic extraction processes using the same fiber feedstock and were found to be almost identical. It was observed that hydroscopic and film forming properties were unaltered.

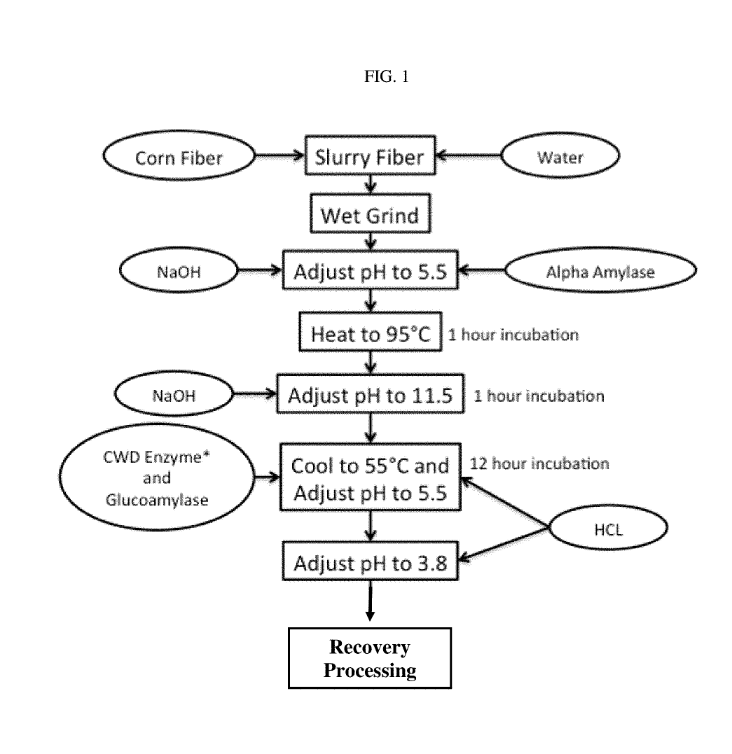

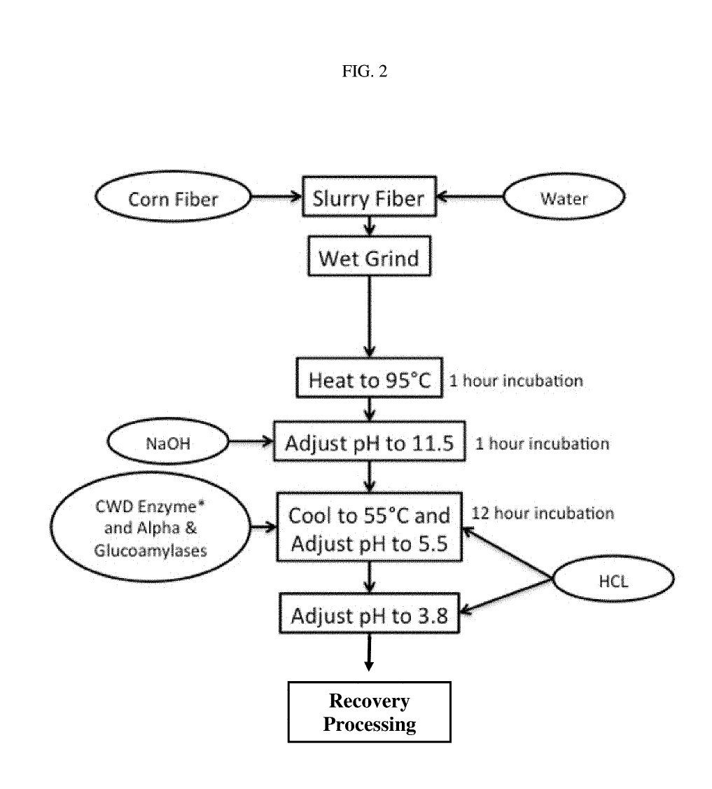

Turning to FIG. 1 and FIG. 2, flowcharts illustrating embodiments of the invention are shown. A bio-based fiber mixture (e.g., labeled “Corn Fiber” with right arrow) is mixed with water to create a slurry, such as a ground slurry or a wet slurry (e.g., labeled “Slurry Fiber”). Corn fiber, for example, can be prepared by corn wet milling processes known in the art where the kernels are separated into germ, fiber, starch, and protein. It can also be prepared by dry milling where the kernel is ground and separated using sizing, density, and air classification equipment to produce germ, pericarp fiber, flour, and grits (e.g., endosperm pieces). In an embodiment, the slurry is subjected to a wet-grinding process (e.g., labeled “Wet-Grind”) to aid in blending and breaking up (e.g., homogenizing or essentially homogenizing) the feedstock for further processing. In other embodiments, dry fiber may be used. Preferably, the dry fiber is ground using any grinding process known in the art. The milling or grinding process is generally performed to increase speed of mixing and decrease particle size. It should be appreciated that other types of grinding could be utilized by a skilled artisan in the methods of the invention. The wet-grinding process typically produces a ground slurry (e.g., an aqueous ground fiber slurry) that is slightly acidic, and pH adjustments are performed with addition of a base, such as NaOH. The pH chosen at this stage is based on the activity range of the particular enzymes to be added and may be selected by a skilled artisan as applicable. It should be appreciated, however, that pH adjustments may be performed with any suitable buffer or pH adjusting agent as selected by a skilled artisan to create a pH-adjusted slurry (e.g., labeled “Adjust pH to 5.5”).

In FIG. 1, the starch-degrading enzymatic process of the present invention requires the ground slurry or the wet slurry to be adjusted to have a pH from about 4.5 to about 6.5 (e.g., 4.5 to 6.5), preferably from about 5 to about 6 (e.g., 5 to 6), more preferably from about 5.1 to about 5.8 (e.g., 5.1 to 5.8), and most preferably from about 5.2 to about 5.5 (e.g., 5.2 to 5.5). In a preferred embodiment, the pH is adjusted to about 5.5 (e.g., 5.5, 4.95 to 6.05, or 5 to 6, or 5.3 to 5.7). A starch-degrading enzymatic component is added to the pH-adjusted slurry, or, alternatively, to the ground slurry prior to pH adjusting to create an enzyme-treated slurry (e.g., labeled “Alpha Amylase” with left arrow). These enzymes include, for example, alpha amylase and glucoamylase. Commercially available examples include SPEZYME® RSL/Alpha/CL (alpha-amylases available from DuPont Industrial Biosciences), OPTIDEX™ L-400 (a glucoamylase available from DuPont Industrial Biosciences), and Liquozyme® SC (an alpha-amylase available from Novozymes). The selected starch-degrading enzymatic preparation may or may not contain additional components (e.g., coenzymes, cofactors, enzyme helpers, or one or more additional enzymes) to stabilize and/or improve the activity of the starch-degrading enzyme. Some commercially available enzymatic preparations have such additional components to increase the efficiency of the starch-degrading enzyme so less enzyme is needed to ensure sufficient degradation of starches. The particular starch-degrading enzyme used may be selected by the skilled artisan to ensure the starches present in the particular bio-based fiber feedstock used is fully solubilized.

The amount of starch-degrading enzyme preparation added is based on the weight of the active enzyme liquid preparation per weight of starch content in the particular fiber feedstock used. The amount of enzyme used is preferably about 0.01 kg (measured as kilograms of liquid enzyme preparation) per metric ton to about 2 kg per metric ton (e.g., 0.01 to 2), more preferably from about 0.1 kg per metric ton to about 1.1 kg per metric ton (e.g., 0.1 to 1.1), and most preferably from about 0.2 kg per metric ton to about 0.5 kg per metric ton (e.g., 0.2 to 0.5). In a preferred embodiment, the amount of starch-degrading enzyme added is about 0.3 kg per metric ton (e.g., 0.3). In terms of units per liquid gram of enzyme preparation, Spezyme RSL, for example, is 20,100 NLC units/gram of enzyme liquid preparation. NLC units of enzyme activity are determined by the rate of starch hydrolysis as reflected in the rate of decrease in iodine-staining capacity (a standard method known in the art). As another example, Spezyme Alpha is 13,700 Alpha Amylase Units (AAU)/gram of enzyme preparation. Enzyme activity is likewise typically determined by the rate of starch hydrolysis as reflected in the rate of decrease in iodine-staining capacity. One AAU of bacterial α-amylase activity is the amount of enzyme required to hydrolyze 10 mg of starch per minute under specified conditions as understood in the art.

In an alternative embodiment, as shown in FIG. 2, the starch-degrading enzyme is added as a cocktail of at least three enzyme types in a subsequent step (as further discussed below). In this embodiment, for sufficient pretreatment of the starches present in the slurry in preparation for the subsequent enzyme cocktail, the slurry must be incubated for a temperature and for a time period to prepare the slurry for enzymatic degradation and create a pretreated slurry (e.g., labeled “Heat to 95° C.—1 hour incubation”). Preferably, this incubation temperature is from about 70° C. to about 100° C. (e.g., 70° C. to 100° C.), more preferably the temperature is from about 80° C. to about 100° C. (e.g., 80° C. to 100° C.), and most preferably the temperature is from about 90° C. to about 100° C. (e.g., 90° C. to 100 ° c). In a preferred embodiment, the temperature is about 95° C. (e.g., 95° C., 85.5° C. to 104.5° C., or 90° C. to 100° C.). The starches are prepared (e.g., gelatinized by cooking) for subsequent enzymatic degradation. The gelatinization temperature is typically at least about 70° C. and increasing the temperature is advantageous as it leads to more completely gelatinized and more quickly hydrolyzed starch. The rate of heating is generally not critical, however, if a high amount of starch is present the viscosity and temperature of the reaction mixture must be sufficient to avoid gelation. The viscosity may be also adjusted at this stage (e.g., by diluting the preparation so the viscosity is low enough to mix in the enzyme(s) and/or via natural viscosity reduction as enzyme(s) is/are added) to ensure sufficient flowability and subsequent enzymatic activity.

Turning back to the embodiment illustrated in FIG. 1, this incubation is performed after addition of the starch-degrading enzyme to create the enzyme-treated slurry. For sufficient degradation of the starches present in the slurry, the enzyme-treated slurry must be incubated at a temperature and for a time period for the starch-degrading enzyme to be active and create an enzyme-degraded slurry (e.g., labeled “Heat to 95° C.—1 hour incubation”). Preferably, this temperature is from about 70° C. to about 100° C. (e.g., 70° C. to 100° C.), more preferably the temperature is from about 80° C. to about 100° C. (e.g., 80° C. to 100° C.), and most preferably the temperature is from about 90° C. to about 100° C. (e.g., 90° C. to 100 ° c). In a preferred embodiment, the temperature is about 95° C. (e.g., 95° C., 85.5° C. to 104.5° C., or 90° C. to 100° C.). The starch-degrading enzyme (e.g., alpha amylase) hydrolyzes the starch once it is gelatinized by cooking. The gelatinization temperature is typically at least about 70° C. and because the starch-degrading enzyme is thermostable it is more active at higher temperatures and increasing the temperature is advantageous as it leads to more completely gelatinized and more quickly hydrolyzed starch. The rate of heating is generally not critical, however, if a high amount of starch is present the starch-degrading enzyme needs to be in the reaction mixture prior to the mixture reaching gelatinization temperature to avoid gelation.

The enzyme-treated slurry must then be incubated for a time sufficient for the activity of the starch-degrading enzyme to degrade the starches to a level adequate to proceed to the subsequent steps in the method. The incubation time is preferably from about 10 min to about 3 hours (e.g., 10 min to 3 hours), more preferably from about 30 min to about 1.5 hours (e.g., 30 min to 1.5 hours), and most preferably from about 45 min to about 1.5 hours (e.g., 45 min to 1.5 hours). In a preferred embodiment, the enzyme-treated slurry is incubated with the starch-degrading enzyme for about 1 hour (e.g., 1 hour, 54 min to 66 min, or 55 min to 65 min). It should be appreciated that a skilled artisan may select the particular incubation period based on the particular feedstock used as well as the particular starch-degrading enzyme or enzyme mixture used. In general, the incubation period is adjusted based on the amount of enzymatic preparation added to the mixture. For example, if lower amounts of enzyme are used, the incubation period would be longer and vice versa.

After the initial incubation with the starch-degrading enzyme as in FIG. 1 (or the initial incubation in the absence of the starch-degrading enzyme in this step as in FIG. 2), the pH of the enzyme-degraded slurry (or, in an alternative, the pretreated slurry) is adjusted as a pretreatment for the subsequent enzymatic incubation to aid in solubilizing components in the solution to make the fibrous mixture more susceptible to the subsequent enzymatic degradation step. In embodiments, the pH is adjusted from about 8 to about 14 (e.g., 8 to 14) to create a pH-adjusted enzyme-degraded slurry, or, in the alternative, a pH-adjusted pretreated slurry, (e.g., labeled “Adjust pH to 11.5-1 hour incubation”). Preferably the pH is adjusted to be from about 9 to about 14 (e.g., 9 to 14), more preferably from about 10 to about 13 (e.g., 10 to 13), and most preferably from about 11 to about 12 (e.g., 11 to 12). In a preferred embodiment, the pH is adjusted to be about 11.5 (e.g., 11.5, 10.35 to 12.65, or 11 to 12). The pH adjustments may be performed with addition of a base, such as NaOH. It should be appreciated, however, that pH adjustments may be performed with any suitable buffer or pH adjusting agent as selected by a skilled artisan to create the pH-adjusted enzyme-degraded slurry (or the pH-adjusted pretreated slurry). The slurry is incubated for an additional time to create an intermediate product preferably from about 10 min to about 120 min (e.g., 10 min to 120 min), more preferably from about 30 min to about 90 min (e.g., 30 min to 90 min), and most preferably from about 50 min to about 70 min (e.g., 50 min to 70 min). In a preferred embodiment, incubation takes place at this stage for about 1 hour (e.g., 1 hour, 54 min to 66 min, or 55 min to 65 min). In general, a longer incubation period may compensate for lower temperature or pH. Preferably, the incubation period is selected to fully solubilize and pretreat the fibrous mixture; however, it should be appreciated that somewhat less than complete solubilization may occur while still allowing to achieve the advantages of the invention.

The composition of the degraded mixture referred to above as the intermediate product consists generally of a soluble portion and an insoluble portion. Much of the protein present in the original fibrous mixture has been denatured, lipids saponified, and the fiber is more extensively hydrated. Overall, the fiber is now less associated with other components allowing improved enzyme access and hydrolysis for the subsequent steps of the method. It should be noted that that the starch-degrading enzyme becomes inactivated by raising the higher pH conditions and is not necessarily removed or isolated from the mixture.

The subsequent step of the method includes an incubation of the intermediate product under different temperature and pH conditions as well as the presence of a cell wall degrading enzyme system to create a cooled intermediate product (e.g., labeled “Cool to 55° C. and Adjust pH to 5.5-12 hour incubation”). The embodiment illustrated in FIG. 2 includes additional enzymes as part of an enzymatic cocktail including the starch-degrading enzyme and optionally glucoamlyase. In alternative embodiments, the temperature and the pH may be adjusted simultaneously or substantially simultaneously, or the temperature may be adjusted prior to the pH adjustment, or the pH may be adjusted prior the temperature adjustment. The pH adjustments may be performed with addition of an acid, such as HCL. It should be appreciated, however, that pH adjustments may be performed with any suitable buffer or pH adjusting agent as selected by a skilled artisan to prepare the cooled intermediate product for the subsequent incubation. The subsequent cell wall degrading enzymatic process of the present invention requires the cooled intermediate product to have a pH adjusted from about 2.5 to about 7.0 (e.g., 2.5 to 7), preferably from about 3.0 to about 6.5 (e.g., 3.0 to 6.5), more preferably from about 3.5 to about 6.0 (e.g., 3.5 to 6.0), and most preferably from about 4.5 to about 5.5 (e.g., 4.5 to 5.5). In a preferred embodiment, the pH is adjusted to about 5.5 (e.g., 5.5, 4.95 to 6.05, or 5 to 6, or 5.3 to 5.7). The particular pH is selected for optimum activity of the enzyme or enzymes being used. The specific pH will generally have minimal effect on the final product, but may impact the amount of enzyme necessary. CWD enzymes (as well as the starch-degrading enzymes and glucoamylase) are normally active in the pH range of about 3 to about 6.5. It is therefore important for the skilled artisan to select the pH matching the optimum activity of the enzymatic preparation being utilized. For example, a change of the particular enzyme applied in this step may necessitate a shift in pH to maintain optimal activity of the selected enzyme. The temperature of the intermediate product is decreased from the previous steps preferably to be from about 10° C. to about 70° C. (e.g., 10° C. to 70° C.), more preferably to be from about 40° C. to about 70° C. (e.g., 40° C. to 70° C.), and most preferably from about 50° C. to about 60° C. (e.g., 50° C. to 60° C.). In a preferred embodiment, the temperature of the pH-adjusted enzyme-degraded ground slurry is about 55° C. (e.g., 55° C., 49.5° C. to 60.5° C., or 50° C. to 60° C.). The CWD enzymes need to be incubated at a temperature high enough that they will have optimum activity but not so as to inactivate the enzymatic preparation. In most cases for CWD enzymes, the optimum temperature is from about 50° C. to about 60° C. It should be appreciated, however, that specific selected enzymes may have higher or lower optimal temperature ranges.

A cell wall degrading enzyme is added either during or after the pH and temperature adjusted as explained above to create a cooled intermediate CWD product. In embodiments, the cell wall degrading enzyme includes one or more enzymes from many different cell wall degrading preparations or mixtures (e.g., labeled “CWD Enzyme and Glucamylase” with right arrow). For example, the cell wall degrading enzyme system may include glucanases, chitinases, xylanases, endocellulases, exocellulases, pectinases, polygalacturonases, the like, and any mixture or combination thereof. Endocellulase or a preparation containing primarily endocellulase with other minor amounts of CWD enzymes is preferred. Examples of commercially available cell wall degrading enzymes include those used in the examples as well as Optimash® (available from DuPont), IndiAge® Super L (available from Genencor). The amount of cell wall degrading enzyme added is preferably about 0.01 kg (measured as kilograms of liquid enzyme preparation) to about 20 kg (e.g., 0.01 kg to 20 kg) per kg total fiber content, more preferably from about 0.1 kg to about 10 kg (e.g., 0.1 kg to 10 kg), and most preferably from about 0.2 kg to about 5 kg (e.g., 0.2 kg to 5 kg). In a preferred embodiment, the amount of cell wall degrading enzyme added is about 0.5 kg (e.g., 0.4 kg, 0.45 kg, 0.5 kg, 0.55 kg, 0.6 kg). In terms of units of enzyme, the GC220 preparation, for example, has 6200 IU/gram of liquid preparation. One IU of activity liberates 1 micro mole of reducing sugar (expressed as glucose equivalents) in one minute from carboxymethylcellulose. In another example, Multifect GC has 82 GCU/gram of liquid preparation. GCU activity measures the amount of glucose released during incubation of a specific type of filter paper as known in the art with the enzyme at 50° C. in a 60 minute period.

In alternative embodiments, the CWD enzyme system of FIG. 1 (or the enzymatic cocktail of FIG. 2) is further combined with a glucoamylase as a precaution to be certain the starch is hydrolyzed to the extent possible for a given fiber source. In such embodiments, the amount of glucoamylase added is preferably about 0.01 kg (measured as kilograms of liquid enzyme preparation) to about 20 kg (e.g., 0.01 kg to 20 kg) per metric ton of starch content of the particular fiber feedstock used, more preferably from about 0.1 kg to about 10 kg (e.g., 0.1 kg to 10 kg), and most preferably from about 0.2 kg to about 5 kg (e.g., 0.2 kg to 5 kg). In a preferred embodiment, the amount of starch-degrading enzyme added is about 0.5 kg (e.g., 0.4 kg, 0.45 kg, 0.5 kg, 0.55 kg, 0.6 kg).

As with other enzymes disclosed herein, the particular amounts of enzyme(s) added in this step may be adjusted. For example, lower amounts of enzyme(s) may be used with an increased incubation period. It should also be appreciated that the optimum amount of enzyme(s) added may also change depending on the particular enzyme(s) selected. The cooled intermediate CWD product is further incubated to create a degraded product. The incubation time for this step is preferably from about 10 min to about 48 hours (e.g., 10 min to 48 hours), more preferably from about 1 hour to about 36 hours (e.g., 1 hour to 36 hours), and most preferably from about 2 hours to about 24 hours (e.g., 2 hours to 24 hours). In a preferred embodiment, the intermediate product is incubated with the cell wall degrading enzyme system for about 12 hours (e.g., 12 hours, 10.5 hours to 13.5 hours, 10 hours to 14 hours, or 1 hours to 13 hours). A skilled artisan may adjust the incubation conditions to ensure sufficient degradation and hydrolysis for the fibers into water-soluble constituents, and, if added, for the glucoamylase to sufficiently convert any remaining starch into glucose.

The order of cooling/pH adjusting/adding CWD enzyme/enzymatic cocktail is important in that if the enzyme is added before cooling or pH adjustment, the enzyme could be inactivated or have its activity significantly reduced. For example, the pH could be adjusted prior to cooling, but the temperature adjustment would preferably be included in the pH calibration for favorable results. Temperature is has an impact on pH calibration so calibrating at the proper temperature may have a significant impact on achieving desired enzymatic conversion.

In embodiments, the pH of the degraded product may be further decreased to aid in product separation and recovery. As previously stated, the pH adjustments may be performed with addition of an acid, such as HCL. It should be appreciated, however, that pH adjustments may be performed with any suitable buffer or pH adjusting agent as selected by a skilled artisan to prepare the degraded product for the subsequent recovery steps. The recovery process of the present invention requires the degraded product to have its pH adjusted (e.g., labeled “Adjust pH to 3.8”) from about 2 to about 7 (e.g., 2 to 7), preferably from about 2.5 to about 6 (e.g., 2.5 to 6), more preferably from about 3.0 to about 5.0 (e.g., 3.0 to 5.0), and most preferably from about 3.5 to about 4.5 (e.g., 3.5 to 4.5). In a preferred embodiment, the pH is adjusted to about 3.8 (e.g., 3.8, 3.4 to 4.2, 3.5 to 4.5, or 3.6 to 4.0). For example, the ideal pH will reduce the solubility of lignins, free fatty acids, and other compounds sufficiently such that they become insoluble and can be removed by centrifugation along with any remaining insoluble fiber material. It is understood by those skilled in the art that many of the undesirable compounds can also be precipitated at low pH levels.

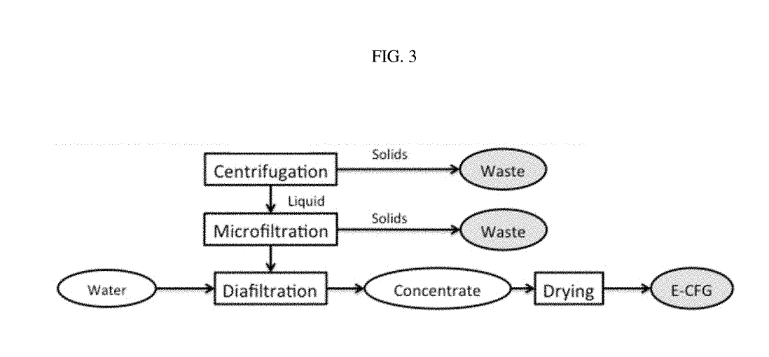

In embodiments, the recovery process includes recovering essentially purified corn fiber gum from the degraded product (e.g., FIG. 3 to FIG. 5). The CFG is essentially separated from non-CFG components that may have remained in the solution. The non-CFG components could be, for example, acid soluble lignins, monosaccharides, short oligosaccharides, peptides, proteins, salts, or other materials that would not precipitate at the reduced pH utilized in the previous step. The recovery process illustrated in FIG. 3 includes centrifuging the degraded product (e.g., labeled “Centrifugation”) to separate into a solid waste portion (e.g., labeled “Solids” next to “Centrifugation” with a right arrow to “Waste”) and a liquid portion (e.g., labeled “Liquid” with a down arrow). The liquid portion may be further processed via microfiltering the liquid portion to further separate into a solid waste portion (e.g., labeled “Solids next to “Microfiltration” with a right arrow to “Waste”) and create a microfiltered product (e.g., labeled “Microfiltration”) and optionally adding water to the microfiltered product for a diafiltering process (e.g., labeled “Diafiltration”). In an alternative embodiment of the recovery process, the microfiltration step is not used. The diafiltered product, in embodiments, is further concentrated (e.g., labeled “Concentrate”) and dried (e.g., labeled “Drying”) to create an essentially purified enzymatically-derived corn fiber gum (e.g., labeled “Drying” with right arrow “E-CFG”).

Recovery of the E-CFG following the pH adjustment of the degraded product can be accomplished utilizing several different processes. FIG. 3, as explained above, shows a process where the pH adjusted liquid stream is subjected to centrifugation so that the majority of insoluble solids can be removed. The solubles (in the liquid stream) can then be subjected to microfiltration to remove any remaining insoluble material that could negatively impact the quality of the final product. The microfiltered liquid stream can then be subjected to diafiltration to reduce the salts content in the solution and may also remove the low molecular weight compounds (e.g., glucose) that may be remaining in the liquid. The diafiltered material can be then subjected to additional concentration utilizing ultrafiltration or water evaporative methods prior to drying and production of the final E-CFG.

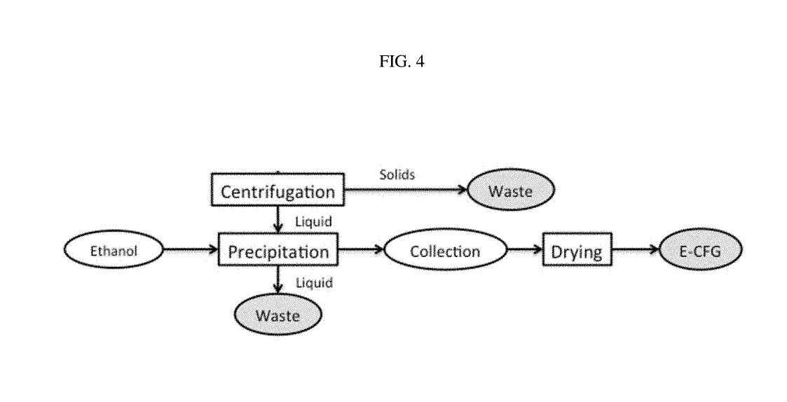

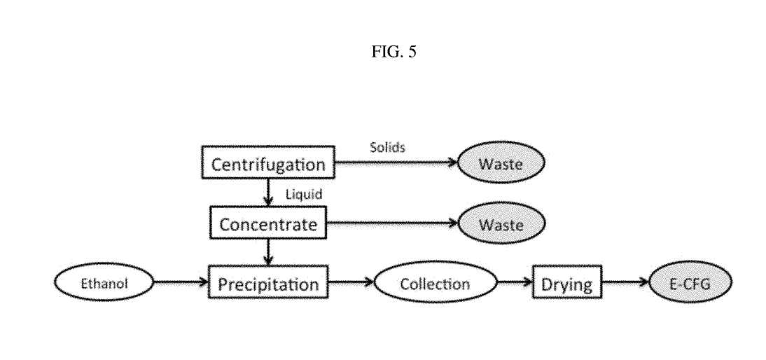

In FIG. 4 and FIG. 5, alternative recovery processing methods are shown where solvent precipitation is utilized. The degraded product is subject to centrifugation (e.g., labeled “Centrifugation”) to separate into a solid waste portion (e.g., labeled “Solids” next to “Centrifugation” with a right arrow to “Waste”) and a liquid portion (e.g., labeled “Liquid” with a down arrow). In FIG. 5, the liquid portion is subject to an optional concentration step (e.g., labelled “Concentration” with a right arrow to “Waste” in FIG. 5) prior to the subsequent precipitation step using, for example, ultrafiltation or evaporative methods as selected by a skilled artisan for a time period sufficient to reduce total volume to a desired level to reduce the amount of solvent used. The “Liquid” portion is subject to precipitation by the addition of a solvent (e.g., lower alcohols such as methanol, ethanol, isopropanol, butanol, etc. as well as water-miscible solvents such as acetone, acetic acid, etc.) which yields a precipitated portion (e.g., labelled “Ethanol” with a right arrow to “Precipitation” followed by “Collection” and “Drying” in FIG. 4 and FIG. 5) and a liquid waste portion (e.g., labelled “Liquid” and “Waste” in FIG. 4). The amount of solvent used is generally from about 1 to about 5 (e.g., 1 to 5) volumes solvent per volume of solution where the volume is dependent on the molecule size (i.e., smaller molecules typically need larger volume addition to cause precipitation as smaller molecules are typically more soluble relative to larger molecules). Precipitation using this method, particularly in the absence of a concentration step, may require the use of increased amounts of solvent and may not be desirable in certain cases as determined by a skilled artisan. The final product (e.g., labelled “E-CFG” in FIG. 4) is then collected. Precipitation is initiated by slowly mixing the solvent into the solution containing the E-CFG. It is important to note that the solvent should be added too rapidly as it could cause localized precipitation and clumping. Reduced temperatures are generally preferred and holding for several hours to maximize product precipitation. Once precipitation is complete, the product is recovered and ideally rinsed with additional solvent to aid in the final drying. The product can then be dried using heat to evaporate any remaining solvent or moisture.

In embodiments, the disclosed invention may also be further be subjected to a hydrolysis process including preparation of an endoxylanase preparation to hydrolyze bio-fiber gum (BFG), which is a commercially available corn bran arabinoxylan product to improve the solubility of the material and clarity of the solutions. Hydrolysates of BFG also have emulsifying ability that was as good as that of the original material, which is already known to have excellent emulsifying ability (U.S. Patent Application Publication No. 2014/0017376; U.S. Patent Application Ser. No. 62/333,456; Yadav, M. P., et al., Journal of Agricultural and Food Chemistry, 56(11): 4181-4187 (2008)). This finding is of a great significance because such functionality is very desirable in the product development context. Coupled with the surprisingly very low viscosity shown by the hydrolysates, their emulsifying ability can potentially allow large amounts of beneficial dietary fiber to be included in food systems where emulsification is required, such as beverages, without the need for including additional emulsifying additives. The enzyme concentration used in the hydrolysis process was seen to have a surprisingly significant effect on the molecular properties and rheological behavior of the hydrolysates. In embodiments, the disclosed invention may also further be processed, in cases where usable water-insoluble fractions may remain, to form a hydrogel for improved performance in applications where the product may be used as an emulsifier (see U.S. patent application Ser. No. 13/768,036).

Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurement. The following examples are intended only to further illustrate the invention and are not intended in any way to limit the scope of the invention as defined by the claims.

### EXAMPLES

### Materials and Methods

Enzymes and Fiber. The following enzymes used were obtained from DuPont Industrial Biosciences: SPEZYME RSL (thermostable alpha-amylase) and OPTIDEX L-400 (glucoamylase). These were used to remove starch from the corn fiber as described below. Cell wall degrading enzymes used were SPEZYME™ CP, GC 220, GC 440, GC 880, Multifect® Xylanase, Multifect® GC, Multifect® GC extra, Accelerase® 1500, Accelerase® XY, Accelerase® XC, and Accelerase® BG. These enzymes were selected for convenience in conducting the described experiments. It should be appreciated that any suitable enzymatic preparation with similar activity may be selected. The fiber used was obtained from a commercial corn wet milling facility. Fiber may generally be obtained from any suitable source, such as, for example, a wet milling facility or a dry milling facility. The fiber used in the experiments herein contained the pericarp and the endosperm fiber from the kernels; however, either pericarp or endosperm or a mixture may be used as disclosed above.

Corn Fiber Gum Extraction. The extraction of CFG was done using a modification to a known procedure (see e.g., Doner, L. W., et al., Isolation and Characterization of Cellulose/Arabinoxylan Residual Mixtures from Corn Fiber Gum Processes, Cereal Chem 2001, 78, 200-204). Corn fiber (30 g) was added to a pre-weighted beaker and 250 g water was added. The fiber was then homogenized using an IKA (Wilmington, N.C.) T25 Disperser with an 18G dispersing element at 10,000 rpm for 3-5 min until slurry was relatively uniform. The probe was rinsed and the slurry moved to a hot plate with a mechanical mixer. The pH was adjusted to 5.5 with 2 M NaOH and alpha amylase (SPEZYME RSL) was added (0.5 mL, about 10,000 NLC units) and the slurry heated to 95° C. for 60 min. The pH was then adjusted to 11.5 with 10 M NaOH and the temperature and pH maintained for 60 min. During heating the beaker was covered to minimize evaporation and water was added as needed to maintain volume of the mixture. The pH was then reduced to 3.8 using 10 N HCL and the mixture cooled overnight at 4° C. to precipitate lignin and other acid insoluble materials. The slurry was transferred to 250 mL centrifuge bottles and centrifuged at 15,000×g for 60 min. The pellet was re-suspended in the same volume of water and centrifuged again. The supernatant from each centrifugation were combined. Hydrogen peroxide is commonly used in such extraction processes to bleach the material; however, it was not used in the disclosed extraction protocol.

CAX Preparation and Enzyme Hydrolysis. The insoluble pellet recovered from the CFG extraction method was wash two additional times with water and recovered by centrifugation. The washed CAX material was re-suspended in 800 mL of water and the slurry pH adjusted to 5.5. While mixing, 10 mL samples were transferred into 15 mL conical bottom test tubes. Enzyme preparations were added to the tubes and incubated at 50° C. overnight. Tubes were centrifuged at 4000×g for 5 min and the pellet volumes determined. Supernatant samples were taken and analyzed by HPLC.

Enzyme-assisted Extraction. The enzyme-assisted extraction of CFG (E-CFG) was done following the same procedure as the recovery of CFG above with the addition of an enzymatic treatment step (illustrated in FIG. 1). Following the de-starching and alkali treatment step described above, the pH was reduced to 5.5 using 10 N HCL. The slurry was transferred to an Erlenmeyer flask and a cell wall degrading enzyme (or enzymes) preparation was added. Glucoamylase (OPTIDEX L-400) was also added (0.2 mL) to hydrolyze any residual dextrins. Dextrins are short chains of glucose typically produced during hydrolysis of starch. If these chains are large enough, but still soluble, they could potentially be recovered with the CFG fraction. This undesirable recovery may be prevented by hydrolyzing any potential dextrins into glucose at this step. It should be appreciated that this is precautionary step and is not mandatory for the effectiveness of the disclosed methods. The flask was then stoppered and a 21 gauge needle inserted for pressure equilibration and incubated at 50° C. for 12 hours. Following incubation, the pH was reduced to 3.8 with 10 N HCL and the contents were transferred to 250 mL centrifuge bottles and centrifuged at 15,000×g for 60 min. The supernatant was collected and used for recovery of the E-CFG.

Filtration of Extracted CFG and E-CFG. The collected extract (and wash, in the case of CFG) was first filtered through two layers of GF/A glass fiber filter using a Buchner funnel and then through Whatman #50 filter paper. This was then transferred to centrifuge bottles and centrifuged again at 10,000×g to remove fine insoluble particles. The recovered supernatant was then filtered through a 0.2 μm filter to remove any remaining insoluble particles. The total volume of filtrate was determined in order to calculate recovery yields.

Ethanol Precipitation and Yield Determination. To recovery the CFG or E-CFG from the extract and for yield determinations, a 100 mL sample of the 0.2 μm filtered extract was transferred to a 500 mL flask with a stir bar. Using constant mixing and a stir plate, 300 mL of absolute ethanol was slowly added. After mixing, the flask was cooled to 4° C. for several hours to allow complete precipitation. Yield was determined by recovering the precipitate on a pre-weighed Whatman #50 filter paper using a Buchner funnel and low vacuum. The precipitate was rinsed several times with absolute ethanol and then the filter paper was removed and dried at 55° C. for several hours. The paper and precipitate was weighted to determine recovery and the precipitate was recovered for further analysis. Total yield was calculated based on recovery and total filtrate volume.

Recovery of the CFG and E-CFG from the extract could alternatively be recovered using diafiltration for salt reduction and ultrafiltration for concentration followed by drying. This process would be the preferred process for large scale processing. The ultrafiltration and diafiltration processes both utilize membranes that allow smaller molecular weight molecules, including water, to pass through the filter while retaining the desired products. In the diafiltration process, an ultrafiltration membrane system is first used for concentrating the CFG or E-CFG and then adding fresh water while continuing to concentrate. This effectively rinses the salt away from the CFG or E-CFG. The concentrated products would therefore be lower in salt as well as lower in concentration of any other molecules that could pass through the membrane. Selection of the optimum molecular weight properties and construction material for the membrane can be accomplished by a skilled artisan.

HPLC and Sugar Analysis. A sub-sample was taken after extraction and/or enzyme treatment and centrifuged at 16,000×g and the supernatant filtered through a 0.2 μm filter (Acrodisc, PALL Life Sciences, Ann Arbor, Mich.). Samples were analyzed using an Agilent 1200 HPLC (Santa Clara, Calif.) as described in Johnston, D. B. & McAloon, A. J., Protease increases fermentation rate and ethanol yield in dry-grind ethanol production. Bioresource Technology 2014, 154, 18-25, with additional sugar calibrations added. All samples were analyzed using Agilent ChemStation software using duplicate injections.

Sugar profiles were determined by hydrolyzing samples in sulfuric acid according to a similar procedure previously described (see e.g., Doner, L. W., et al., Isolation and Characterization of Cellulose/Arabinoxylan Residual Mixtures from Corn Fiber Gum Processes, Cereal Chem 2001, 78, 200-204). The samples were then analyzed for monosaccharides by HPLC.

### Results and Discussion

CAX Hydrolysis. To determine what enzyme or enzymes would work to reduce water binding of the insoluble material and aid in CFG extraction, a slightly modified process (see e.g., see e.g., Doner, L. W., et al., Isolation and Characterization of Cellulose/Arabinoxylan Residual Mixtures from Corn Fiber Gum Processes, Cereal Chem 2001, 78, 200-204) was used to extract CFG and recover the insoluble CAX material. The CAX binds a substantial amount of water. It was washed to remove salt and other solubles from the extraction process by centrifugation and mixed with deionized water to produce a slurry that was uniform for conducing enzymatic treatment tests and the pH was adjusted to 5.5. The solids content of the slurry was determined by dry weight analysis to be 1.35% total solids.

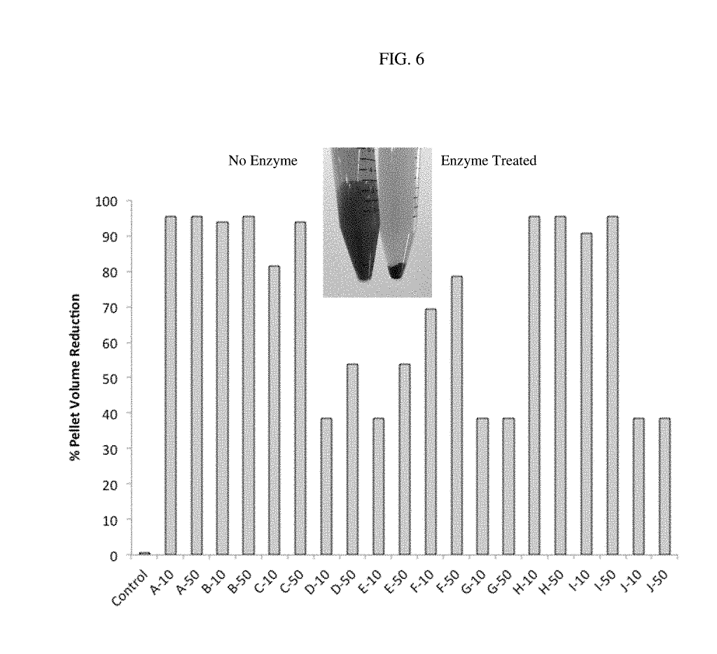

The slurry was distributed into 15 mL test tubes, and mixed with a range of cell wall degrading preparations. Following incubation with the enzyme preparations at 50° C. for 16 hours, the tubes were centrifuged and the pellet volumes recorded. FIG. 6 shows the reduction in pellet volume as a percentage of the enzyme free control that was incubated under the same conditions. Enzymes were added at two different doses (10 and 50 μL/10 mL slurry) and together with the long incubation time were intended to determine if the specific enzyme preparation had activity on the substrate.

The data unexpectedly and surprisingly showed that several enzyme preparations were capable of significantly reducing the pellet volume relative to the control without any added enzyme. The photo inset in FIG. 6 demonstrates how unexpectedly substantial the pellet volume was decreased in the present of enzyme addition. Visual analysis showed that the apparent viscosities of the slurries were also noticeably reduced for many of the enzyme treatments; however, this viscosity observation was not quantified. The unexpected and surprising reduction in pellet volume indicated alteration of the water binding either through solubilizing the CAX or water binding alterations of the CAX fiber. This reduction significantly aided in the recovery of increased amounts of CFG by eliminating the need to do extensive washings of the highly hydrated insoluble material.

Samples of the supernatant were analyzed by HPLC for sugars and for higher molecular weight material. The data is shown in (Table 1) and illustrates that there are significant amounts of material being solubilized by the enzyme treatments. The total solubles measured by HPLC were found to correlate with the pellet volume reduction and likely gave a more quantitative measure of solubilization. The HPLC data also indicates that there are distinct distributions of the solubilized material. In most cases it is either monosaccharide or it is a mixture of both polysaccharide and monosaccharides. Several of the enzyme treatments showed a disaccharide peak believed to be cellobiose at the 10 μL dose; however, this peak was not present at the higher dose. This conversion was likely due to cellobiose being converted into glucose.

TABLE 1
Concentration of sugars released during enzymatic treatments of
the CAX residue produced from corn fiber gum extraction

| | Concentrations (% w/v) | | | | | | |
|---|---|---|---|---|---|---|---|
| Enzyme^{a} | DP4+ | DP3 | DP2 | Glucose | Xylose | Arabinose⁺ | Total |
| Control | 0.024 | 0.000 | 0.000 | 0.000 | 0.000 | 0.005 | 0.03 |
| A-10 | 0.424 | 0.026 | 0.000 | 0.534 | 0.022 | 0.046 | 1.05 |
| A-50 | 0.420 | 0.020 | 0.000 | 0.542 | 0.043 | 0.135 | 1.16 |
| B-10 | 0.395 | 0.025 | 0.160 | 0.379 | 0.037 | 0.048 | 1.04 |
| B-50 | 0.379 | 0.025 | 0.010 | 0.535 | 0.056 | 0.118 | 1.12 |
| C-10 | 0.327 | 0.021 | 0.005 | 0.517 | 0.013 | 0.009 | 0.89 |
| C-50 | 0.400 | 0.023 | 0.000 | 0.547 | 0.027 | 0.021 | 1.02 |
| D-10 | 0.355 | 0.012 | 0.005 | 0.075 | 0.034 | 0.087 | 0.57 |
| D-50 | 0.364 | 0.015 | 0.000 | 0.101 | 0.049 | 0.263 | 0.79 |
| E-10 | 0.358 | 0.007 | 0.002 | 0.052 | 0.028 | 0.057 | 0.50 |
| E-50 | 0.382 | 0.012 | 0.000 | 0.067 | 0.046 | 0.164 | 0.67 |
| F-10 | 0.320 | 0.027 | 0.022 | 0.296 | 0.038 | 0.080 | 0.78 |
| F-50 | 0.238 | 0.029 | 0.000 | 0.491 | 0.092 | 0.102 | 0.95 |
| G-10 | 0.358 | 0.006 | 0.009 | 0.081 | 0.015 | 0.022 | 0.49 |
| G-50 | 0.352 | 0.007 | 0.025 | 0.190 | 0.038 | 0.040 | 0.65 |
| H-10 | 0.433 | 0.026 | 0.000 | 0.548 | 0.028 | 0.054 | 1.09 |
| H-50 | 0.420 | 0.020 | 0.000 | 0.551 | 0.047 | 0.139 | 1.18 |
| I-10 | 0.393 | 0.025 | 0.135 | 0.415 | 0.043 | 0.061 | 1.07 |
| I-50 | 0.391 | 0.028 | 0.000 | 0.546 | 0.061 | 0.168 | 1.19 |
| J-10 | 0.341 | 0.008 | 0.003 | 0.052 | 0.033 | 0.058 | 0.49 |
| J-50 | 0.359 | 0.012 | 0.000 | 0.066 | 0.050 | 0.155 | 0.64 |

^{a}Enzymes were used at 10 and 50 μL with 0.135 g of cellulosic residue in 10 mL at pH 5.5. Letters represent enzyme used and number is the dosage: GC 220 (A), Multifect GC (B), Accellerase 1500 (C), GC 440 (D), Accellerase XY (E), Accellerase XC (F), Accellerase BG (G), GC Extra (H), Spezyme CP (I), and Multifect Xylanase (J). Data shown are the average of duplicate determinations.
⁺Arabinose was combined with other low level sugars, as they were not fully resolved in this separation system.

The HPLC data also presented a surprising result related to free sugars. It had been anticipated that the enzyme treatment of the insoluble material would create a range of monosaccharides; however, glucose was the predominant sugar detected in several enzyme treatments. The remaining material was higher molecular weight material that eluted at the void of the column (DP4+). The void volume is the same location that CFG was found to elute with this column system. The presence of glucose as the predominant monosaccharide indicates that only the cellulose or another non-starch glucan was being hydrolyzed. Other preparations did show the anticipated mixture of monosaccharides indicating a more complete hydrolysis and likely a reduced molecular weight polysaccharide present in the DP4+ peak.

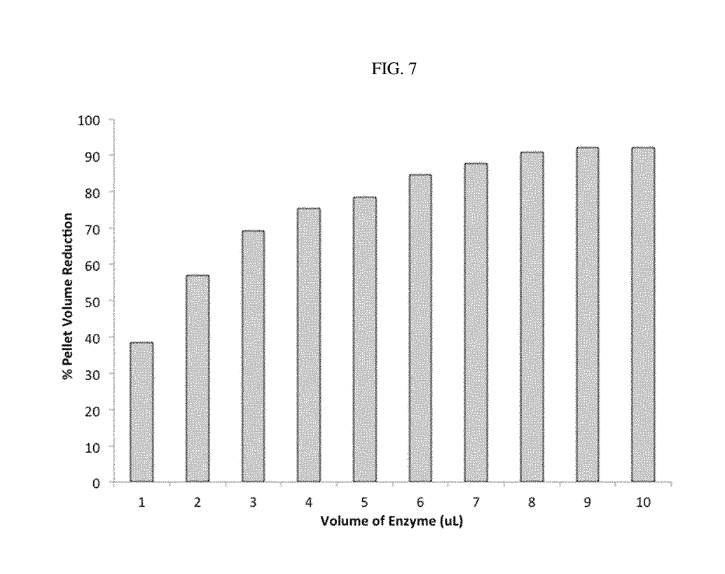

A series of tubes containing CAX were tested with lower levels (1-10 μL) of GC 220 at a fixed 6-hour incubation. This enzyme previously showed almost no production of monosaccharides other than glucose in the first study. FIG. 7 shows the data for the pellet from these hydrolysis experiments. As the dose increased the pellet volume surprisingly decreased. The lowest dose (1 μL) gave a 38% reduction in the pellet volume whereas the highest dose (10 μL) gave a 92% reduction. The overnight incubation at this dose gave a 95% reduction with no further reduction at higher enzyme dose. HPLC analysis of the supernatant (Table 2) showed at the lower enzyme concentrations an increase in disaccharide and an increase in glucose. As the concentrations increase the disaccharide level began to decrease and the glucose levels continued to increase. This information, together with the higher enzyme dosing data, indicates that the disaccharides were being converted into just glucose.

TABLE 2
Concentration of sugars released from CAX residue using GC 220

| Enzyme^{a} | Concentrations (% w/v) | | | | | | |
|---|---|---|---|---|---|---|---|
| (μL) | DP4+ | DP3 | DP2 | Glucose | Xylose | Arabinose⁺ | Total |
| 1 | 0.370 | 0.011 | 0.117 | 0.093 | 0.002 | 0.012 | 0.605 |
| 2 | 0.015 | 0.171 | 0.177 | 0.004 | 0.014 | 0.034 | 0.415 |
| 3 | 0.416 | 0.017 | 0.203 | 0.210 | 0.006 | 0.018 | 0.870 |
| 4 | 0.431 | 0.019 | 0.204 | 0.268 | 0.007 | 0.021 | 0.951 |
| 5 | 0.435 | 0.022 | 0.184 | 0.321 | 0.009 | 0.025 | 0.996 |
| 6 | 0.428 | 0.021 | 0.170 | 0.351 | 0.010 | 0.029 | 1.010 |
| 7 | 0.425 | 0.021 | 0.156 | 0.372 | 0.012 | 0.034 | 1.019 |
| 8 | 0.425 | 0.020 | 0.131 | 0.405 | 0.014 | 0.035 | 1.029 |
| 9 | 0.433 | 0.020 | 0.127 | 0.416 | 0.015 | 0.039 | 1.049 |
| 10 | 0.424 | 0.020 | 0.108 | 0.437 | 0.016 | 0.041 | 1.045 |

^{a}GC 220 added to 0.135 g of cellulosic residue in 10 mL at pH 5.5.
⁺Arabinose was combined with other low level sugars, as they were not fully resolved in this separation system. Data shown are the averages of duplicate determinations.

CAX to CFG. Although the data was not conclusive, it was believed that the hydrolysis of the CAX by some enzyme preparations was releasing additional CFG. These results could indicate that the water binding properties of the CAX and the hydroscopic properties of the CFG are due to similar functional groups being present. The insoluble CAX is potentially comprised of CFG-like molecules attached to an insoluble cellulosic backbone and the CFG is a soluble version of a similar molecule.

To test this hypothesis, the now soluble molecules were isolated from the enzymatic hydrolysis mixture of one of the enzyme treatments by filtering and then precipitating with 3 volumes of ethanol. The recovered material was analyzed for sugar profile and compared with the sugar profiles of CFG isolated without cell wall degrading treatments. The sugar profiles are shown in Table 3.

TABLE 3
Fraction of total sugars from Hydrolysis of isolated fractions

| | Fraction of Total Sugar (%) | | | | |
|---|---|---|---|---|---|
| Sample | Glucose | Xylose | Galactose | Arabinose | Ara/Xyl |
| Z-Trim ^{a} | 41.58 | 29.75 | 11.32 | 17.35 | 0.583 |
| CFG ^{a} | 0.00 | 47.64 | 16.92 | 35.45 | 0.744 |
| CFG | 2.95 | 43.07 | 16.24 | 37.74 | 0.876 |
| E-CFG | 2.29 | 43.44 | 17.06 | 37.21 | 0.857 |
| E-CAX | 1.49 | 43.35 | 18.62 | 36.54 | 0.843 |
| Hyd. Z-Trim ^{b} | 1.20 | 45.82 | 19.93 | 33.06 | 0.722 |

^{a} Samples were obtained from AgriTech Worldwide (formerly Z-Trim).
^{b} Hydrolyzed Z-Trim was prepared by treating Z-Trim with a cell wall degrading enzyme and recovering the soluble polysaccharide produced using 3x ethanol precipitation.

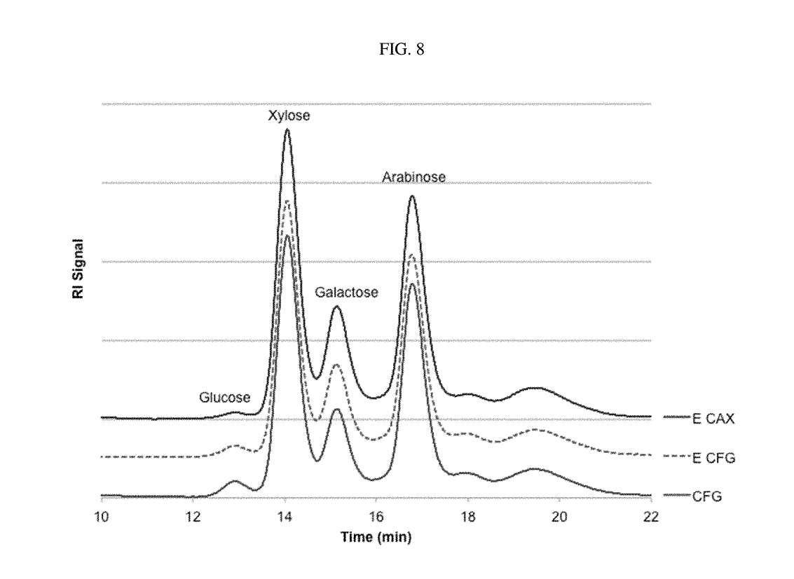

The sugar compositional data shows that the enzymatic-released material is almost identical in sugar profile to the CFG. Additionally, the ethanol-isolated material contains similar hydroscopic properties to CFG and was found to form a film on drying like CFG as well. FIG. 8 shows an overlay of the chromatography used for the monosaccharide analysis quantified in Table 3 in order to demonstrate the similarities of the sugar profiles.

Enzymatic Corn Fiber Gum (E-CFG) Extraction. Adjusting the CFG extraction process, the enzyme treatment step was incorporated as described in the methods section and outlined in FIG. 1 and FIG. 2. The addition of a glucoamylase for more complete starch conversion and to filtration improvement was also incorporated. Additionally, a pH reduction to remove both hemi-A and other insoluble material in the same step was done prior to corn fiber gum recovery to simplify the overall process.

HPLC comparison of the extracts (before ethanol precipitation) showed both, CFG and E-CFG, had the majority of material eluting as polysaccharides; however, the extract of the E-CFG also had a glucose peak representing about 15% of the total eluted material. The glucose was produced from starch with the glucoamylase as well as with the cell wall degrading enzyme. The CFG extract did not have a glucose peak detectable, potentially indicating the hydrolyzed starch was still eluting in the DP4+ region of the chromatogram.

Yield comparison, by ethanol precipitation, with the enzyme-assisted extraction (E-CFG) process surprisingly showed a 19.8% increase in recovery relative to the CFG process without the use of enzymes. Additionally, the E-CFG process surprisingly produced a more concentrated extract, as washing of the pellet was no longer necessary. The increased concentration allows an overall reduction in the amount of processing water needed.

The functional properties of the E-CFG were not fully tested in this study but sufficient evidence was generated to conclude that incorporating the enzyme extraction did not significantly alter functionality of the arabinoxylan product. The E-CFG was found to be highly hydroscopic, which is a property of conventional corn fiber gum. E-CFG was also found to form films similar to conventional CFG upon oven drying of a solution.

The examples demonstrate that a highly soluble, functional corn fiber gum may be produced from corn fiber with surprisingly and significantly improved yields over conventional processes. Improved recovery of CFG was demonstrated through the enzymatic processing of CAX, to surprisingly illustrate that the method of the invention simultaneously decreases solid waste products and increases industrially valuable corn fiber gum. Multiple enzyme preparations were evaluated for improved yields of corn fiber gum, where incorporating the enzyme treatment of the invention, corn fiber gum yields were surprisingly improved relative to the conventional non-enzymatic processes commonly used in industry.

Therefore, this disclosure relates to compositions comprising a bio-based fiber gum product subjected to an enzymatic process wherein an insoluble fraction of the bio-based fiber gum product is reduced by at least about 35% or at least about 50% as compared to the bio-based fiber gum product not subjected to the enzymatic process optionally comprising at least one enzyme selected from the group consisting of: a starch-degrading enzyme and a cell-wall degrading enzyme.

This disclosure further relates a process for producing a bio-based fiber gum optionally selected from the group consisting of oat fiber gum, corn fiber gum, sorghum fiber gum, wheat fiber gum, and combinations thereof from a bio-based fiber feedstock, the process comprising: (a) subjecting the bio-based fiber feedstock to a process to create a slurry; (b) either (i) adjusting the pH of the slurry to create a pH-adjusted slurry or (ii) heating the slurry without adjusting the pH of the slurry to create a heated slurry; (c) adding a starch-degrading enzymatic component to the pH-adjusted slurry to create an enzyme-treated slurry; (d) either (i) incubating the enzyme-treated slurry at a temperature and time sufficient to create an enzyme-degraded slurry or (ii) incubating the heated slurry at a temperature and time sufficient to pretreat the heated slurry to create an pretreated slurry; (e) adjusting the pH of (i) the enzyme-degraded slurry or (ii) the pretreated slurry to create (i) a pH-adjusted enzyme-degraded slurry or (ii) a pH-adjusted pretreated slurry; (f) incubating (i) the pH-adjusted enzyme-degraded slurry or (ii) the pH-adjusted pretreated slurry to create an intermediate product; (g) cooling the intermediate product to create a cooled intermediate product; (h) adding a cell wall degrading (CWD) enzyme system and optionally glucoamylase to the cooled intermediate product to create a cooled intermediate CWD product; and (i) incubating the cooled intermediate CWD product to create a degraded product.

This disclosure also relates to process for producing a bio-based fiber gum from a bio-based fiber feedstock, the process comprising: (a) subjecting the bio-based fiber feedstock to a process to create a slurry; (b) pretreating the slurry by (i) heating the slurry and (ii) incubating the slurry to create a pretreated slurry; (c) adjusting the pH of the pretreated slurry to create a pH-adjusted pretreated slurry; (d) adding an enzymatic cocktail comprising at least one amylase, at least one cell wall degrading enzyme, and optionally glucoamylase to the pH-adjusted pretreated slurry to create an enzymatic cocktail-treated slurry; (e) incubating the enzymatic cocktail-treated slurry to create an intermediate product; (f) adjusting the pH of the intermediate product; and (g) recovering the bio-based fiber gum

While this invention may be embodied in many different forms, there are described in detail herein specific preferred embodiments of the invention. The present disclosure is an exemplification of the principles of the invention and is not intended to limit the invention to the particular embodiments illustrated. All patents, patent applications, scientific papers, and any other referenced materials mentioned herein are incorporated by reference in their entirety, including any materials cited within such referenced materials. Furthermore, the invention encompasses any possible combination of some or all of the various embodiments and characteristics described herein and/or incorporated herein. In addition the invention encompasses any possible combination that also specifically excludes any one or some of the various embodiments and characteristics described herein and/or incorporated herein.

The amounts, percentages and ranges disclosed herein are not meant to be limiting, and increments between the recited amounts, percentages and ranges are specifically envisioned as part of the invention. All ranges and parameters disclosed herein are understood to encompass any and all subranges subsumed therein, and every number between the endpoints. For example, a stated range of “1 to 10” should be considered to include any and all subranges between (and inclusive of) the minimum value of 1 and the maximum value of 10 including all integer values and decimal values; that is, all subranges beginning with a minimum value of 1 or more, (e.g., 1 to 6.1), and ending with a maximum value of 10 or less, (e.g. 2.3 to 9.4, 3 to 8, 4 to 7), and finally to each number 1, 2, 3, 4, 5, 6, 7, 8, 9, and 10 contained within the range.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. As used herein, the term “about” refers to a quantity, level, value, or amount that varies by as much as 30%, preferably by as much as 20%, and more preferably by as much as 10% to a reference quantity, level, value, or amount.

The term “consisting essentially of” excludes additional method (or process) steps or composition components that substantially interfere with the intended activity of the method (or process) or composition. This term may be substituted for inclusive terms such as “comprising” or “including” to more narrowly define any of the disclosed embodiments or combinations/sub-combinations thereof. Furthermore, the exclusive term “consisting” is also understood to be substitutable for these inclusive terms.

“Optional” or “optionally” means that the subsequently described event or circumstance may or may not occur, and that the description includes instances in which said event or circumstance occurs and instances where it does not. For example, the phrase “optionally comprising a defoaming agent” means that the composition may or may not contain a defoaming agent and that this description includes compositions that contain and do not contain a foaming agent.

By the term “effective amount” of a compound or property as provided herein is meant such amount as is capable of performing the function of the compound or property for which an effective amount is expressed. As is pointed out herein, the exact amount required will vary from process to process, depending on recognized variables such as the compounds employed and various internal and external conditions observed as would be interpreted by one of ordinary skill in the art. Thus, it is not possible to specify an exact “effective amount,” though preferred ranges have been provided herein. An appropriate effective amount may be determined, however, by one of ordinary skill in the art using only routine experimentation.

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims. Although any methods and materials similar or equivalent to those described herein can be used in the practice or testing of the present invention, the preferred methods and materials are herein described. Those skilled in the art may recognize other equivalents to the specific embodiments described herein which equivalents are intended to be encompassed by the claims attached hereto.

The claimed invention is:

1. A composition comprising: a bio-based fiber gum product subjected to an enzymatic process wherein an insoluble fraction of the bio-based fiber gum product is reduced by at least about 35% as compared to the bio-based fiber gum product not subjected to the enzymatic process.

2. The composition of claim 1, wherein the enzymatic process comprises at least one enzyme selected from the group consisting of: a starch-degrading enzyme and a cell-wall degrading enzyme.

3. The composition of claim 1, wherein the insoluble fraction of the bio-based fiber gum product is reduced by at least about 50% as compared the bio-based fiber gum product not subjected to the enzymatic process.

4. A process for producing a bio-based fiber gum from a bio-based fiber feedstock, the process comprising: (a) subjecting the bio-based fiber feedstock to a process to create a slurry; (b) either (i) adjusting the pH of the slurry to create a pH-adjusted slurry or (ii) heating the slurry without adjusting the pH of the slurry to create a heated slurry; (c) adding a starch-degrading enzymatic component to the pH-adjusted slurry to create an enzyme-treated slurry; (d) either (i) incubating the enzyme-treated slurry at a temperature and time sufficient to create an enzyme-degraded slurry or (ii) incubating the heated slurry at a temperature and time sufficient to pretreat the heated slurry to create an pretreated slurry; (e) adjusting the pH of (i) the enzyme-degraded slurry or (ii) the pretreated slurry to create (i) a pH-adjusted enzyme-degraded slurry or (ii) a pH-adjusted pretreated slurry; (f) incubating (i) the pH-adjusted enzyme-degraded slurry or (ii) the pH-adjusted pretreated slurry to create an intermediate product; (g) cooling the intermediate product to create a cooled intermediate product; (h) adding a cell wall degrading (CWD) enzyme system and optionally glucoamylase to the cooled intermediate product to create a cooled intermediate CWD product; and (i) incubating the cooled intermediate CWD product to create a degraded product.

5. The process of claim 4, wherein the bio-based fiber feedstock is selected from the group consisting of: oat fiber, corn fiber, sorghum fiber, wheat fiber, and combinations thereof.

6. The process of claim 4, wherein the bio-based fiber gum is corn fiber gum.

7. The process of claim 4, comprising subjecting the bio-based fiber feedstock to a wet grind process to create the slurry.

8. The process of claim 4, comprising adjusting the pH of the slurry to be from about 4.5 to about 6.5.

9. The process of claim 4, wherein the starch-degrading enzyme is selected from the group consisting of: an alpha-amylase, a glucoamylase, and combinations thereof.

10. The process of claim 4, wherein from about 0.01 kg to about 2 kg of active starch-degrading enzyme liquid preparation is added per metric ton of starch in the bio-based fiber feedstock.

11. The process of claim 4, wherein incubating the enzyme-treated slurry at the temperature and time sufficient to create the enzyme-degraded slurry comprises incubating the enzyme-treated slurry at a temperature from about 70° C. to about 100° C.

12. The process of claim 4, wherein incubating (i) the enzyme-treated slurry or (ii) the heated slurry at the temperature and time sufficient to create (i) the enzyme-degraded slurry or (ii) the pretreated slurry comprises incubating (i) the enzyme-treated slurry or (ii) the heated slurry for a period from about 10 min to about 3 hours.

13. The process of claim 4, wherein adjusting the pH of (i) the enzyme-degraded slurry or (ii) the pretreated slurry to create (i) the pH-adjusted enzyme-degraded slurry or (ii) the pH-adjusted pretreated slurry comprises adjusting the pH of (i) the enzyme-degraded slurry or (ii) the pretreated slurry to be from about 8 to about 14.

14. The process of claim 4, wherein incubating the pH-adjusted enzyme-degraded slurry comprises a period from about 10 min to about 120 min.

15. The process of claim 4, comprising cooling the intermediate product to a temperature from about 10° C. to about 70° C.

16. The process of claim 4, comprising adjusting the pH of the intermediate product from about 2.5 to about 7.

17. The process of claim 4, wherein the cell wall degrading enzyme system is selected from the group consisting of: glucanases, chitinases, xylanases, endocellulases, exocellulases, pectinases, polygalacturonases, starch-degrading enzymes, and any mixture or combination thereof.

18. The process of claim 4, wherein from about 0.01 kg to about 20 kg of active cell wall degrading enzyme liquid preparation is added per metric ton of starch in the bio-based fiber feedstock.

19. The process of claim 4, wherein from about 0.01 kg to about 20 kg of active glucoamylase liquid preparation is added per metric ton of starch in the bio-based fiber feedstock.

20. The process of claim 4, comprising incubating the cooled intermediate CWD product from about 10 min to about 48 hours.

21. The process of claim 4, further comprising decreasing the pH of the degraded product to be from about 2 to about 7.

22. The process of claim 4, further comprising recovering essentially purified corn fiber gum from the degraded product.

23. The process of claim 4, further comprising (i) centrifuging the degraded product to separate the degraded product into a solid waste portion and a liquid portion, (ii) microfiltering the liquid portion to create a microfiltered product, (iii) optionally adding water to the microfiltered product, (iv) diafiltering the microfiltered product to create a diafiltered product, (v) concentrating and drying the diafiltered product, (vi) recovering essentially purified bio-based fiber gum.

24. A process for producing a bio-based fiber gum from a bio-based fiber feedstock, the process comprising: (a) subjecting the bio-based fiber feedstock to a process to create a slurry; (b) pretreating the slurry by (i) heating the slurry and (ii) incubating the slurry to create a pretreated slurry; (c) adjusting the pH of the pretreated slurry to create a pH-adjusted pretreated slurry; (d) adding an enzymatic cocktail comprising at least one amylase, at least one cell wall degrading enzyme, and optionally glucoamylase to the pH-adjusted pretreated slurry to create an enzymatic cocktail-treated slurry; (e) incubating the enzymatic cocktail-treated slurry to create an intermediate product; (f) adjusting the pH of the intermediate product; and (g) recovering the bio-based fiber gum.
